(19) United States
(12) Patent Application Publication
(10) Pub. No.: US 2017/0189047 A1
(43) Pub. Date: Jul. 6, 2017

(54) Lumpectomy Knife Set

(71) Applicant: Firas Eladoumikdachi, Princeton, NJ (US)

(72) Inventor: Firas Eladoumikdachi, Princeton, NJ (US)

(21) Appl. No.: 15/394,030

(22) Filed: Dec. 29, 2016

Related U.S. Application Data
(60) Provisional application No. 62/275,308, filed on Jan. 6, 2016.

(51) Int. Cl. A61B 17/3205

(52) U.S. Cl. CPC A61B 17/3205; A61B 2017/00473

(57) ABSTRACT

A lumpectomy knife set including a main knife having at least one first planar wall extending between an upper edge and a knife edge with the at least one first planar wall defining a first geometric shape about a first through opening. A margin knife has at least one second planar wall extending between an upper edge and a knife edge with the at least one second planar wall defining a second geometric shape about a second through opening larger than the first through opening. The second geometric shape complements the first geometric shape such that the margin knife is concentrically positionable about the main knife with a margin gap between the main knife and the margin knife.

### CROSS-REFERENCE TO RELATED APPLICATIONS

This application claims the benefit of U.S. Provisional Application No. 62/275,308, filed Jan. 6, 2016, which is incorporated by reference in its entirety herein.

### TECHNICAL FIELD

This present disclosure relates to a lumpectomy cutting device, and more particularly, to a multi-dimension, two-unit knife set for lumpectomy procedures and similar procedures.

### BACKGROUND

Lumpectomy is a surgical procedure that involves removing a suspected malignant (cancerous) tumor, or lump, and a small portion of the surrounding tissue from a woman's breast. The surgeon's goal during surgery is to take out all of the cancer along with a rim of normal tissue around it. During or after surgery, a pathologist examines this rim of tissue—called the surgical margin or margin of resection—to be sure it is clear of any cancer cells. If cancer cells are present, this will influence decisions about treatments such as additional surgery and radiation.

Typically, the surgeon utilizes a scalpel to cut around the target tissue and the rim of normal tissue. Conventional scalpels require multiple slicing strokes, which approaches carving that can leave rough or jagged edges. While some current scalpels slice nicely, they still leave ‘train tracks’ (the narrow incisions), which make margin assessment inaccurate, and unnecessarily leave undesired cuts. Moreover, current techniques using multiple splicing movements around all sides of the tumor using available scalpels create a challenge for properly orienting the excised specimen, thus causing inaccurate margin assessment.

### SUMMARY

In at least one embodiment, the present disclosure provides a lumpectomy knife set including a main knife having at least one first planar wall extending between an upper edge and a knife edge with the at least one first planar wall defining a first geometric shape about a first through opening. A margin knife has at least one second planar wall extending between an upper edge and a knife edge with the at least one second planar wall defining a second geometric shape about a second through opening larger than the first through opening. The second geometric shape complements the first geometric shape such that the margin knife is concentrically positionable about the main knife with a margin gap between the main knife and the margin knife.

In at least one embodiment, the first and geometric shapes are each selected from one of square, rectangular, oval and circular.

In at least one embodiment, the main knife and the margin knife include index markings to facilitate concentric alignment therebetween.

In at least one embodiment, the main knife and the margin knife are interconnected to one another by connectors extending within the margin gap.

In at least one embodiment, the main knife and the margin knife are interconnected to one another by a handle assembly.

In at least one embodiment, the handle assembly includes a handle member extending between a pair of connection platforms such that each of the connection platforms includes a plurality of clips extending therefrom with spaces therebetween to receive the upper edges of the main knife and the margin knife.

In at least one embodiment, the at least one first planar wall of the main knife has a convex knife edge and the at least one second planar wall of the margin knife has a convex knife edge.

In at least one embodiment, the knife edges are interchangeable.

In at least one embodiment, the main knife includes a main body having a geometric shape defined by a plurality of walls configured to extend between an upper edge and a lower connector edge such that the lower connector edge is configured for connection to a blade assembly.

In at least one embodiment, the blade assembly includes a plurality of walls which complement the shape of the main body with each wall having a connector edge and a knife edge such that the connector edge of each wall is configured to removably connect with the connector edge of the main body through a snap-fit connection.

In at least one embodiment, the blade assembly includes a plurality of walls which complement the shape of the main body with each wall having a connector edge in which two of the walls have a convex knife edge and the other walls have a linear knife edge such that the connector edge of each wall is configured to removably connect with the connector edge of the main body through a snap-fit connection.

### BRIEF DESCRIPTION OF THE DRAWINGS

The accompanying drawings, which are incorporated herein and constitute part of this specification, illustrate the presently preferred embodiments of the disclosure, and, together with the general description given above and the detailed description given below, serve to explain the features of the disclosure. In the drawings:

FIG. 1 is an exploded perspective view of a knife set in accordance with an embodiment of the present disclosure.

FIG. 2 is a top view of the assembled knife set of FIG. 1.

FIG. 3 is a top view of a knife set in accordance with another embodiment of the present disclosure.

FIG. 4 is a top view of a knife set in accordance with another embodiment of the present disclosure.

FIG. 5 is a perspective view of the main knife of the knife set of FIG. 4.

FIG. 6 is a cross-sectional view along the line 6-6 in FIG. 1.

FIG. 7 is a perspective view of a main knife of another embodiment of the present disclosure.

FIG. 8 is a perspective view of a main knife of yet another embodiment of the present disclosure.

FIG. 9 is an exploded perspective view of a main knife of yet another embodiment of the present disclosure with an interchangeable knife edge.

FIG. 10 is an exploded perspective view of the main knife of FIG. 9 with a different interchangeable knife edge.

FIG. 11 is an exploded front elevation view of the knife set of FIG. 1 with an optional handle member.

### DETAILED DESCRIPTION

In consideration of the figures, it is to be understood for purposes of clarity that certain details of construction and/or operation are not provided in view of such details being conventional and well within the skill of the art upon disclosure of the document described herein. In the drawings, like numerals indicate like elements throughout. Certain terminology is used herein for convenience only and is not to be taken as a limitation of the present disclosure. The following describes preferred embodiments of the present disclosure. However, it should be understood that the present disclosure is not limited by the preferred embodiments described herein.

As used in this document, the singular forms “a,” “an,” and “the” include plural reference unless the context clearly dictates otherwise. Unless defined otherwise, all technical and scientific terms used herein have the same meanings as commonly understood by one of ordinary skill in the art. As used herein, the term “comprising” means “including, but not limited to.”

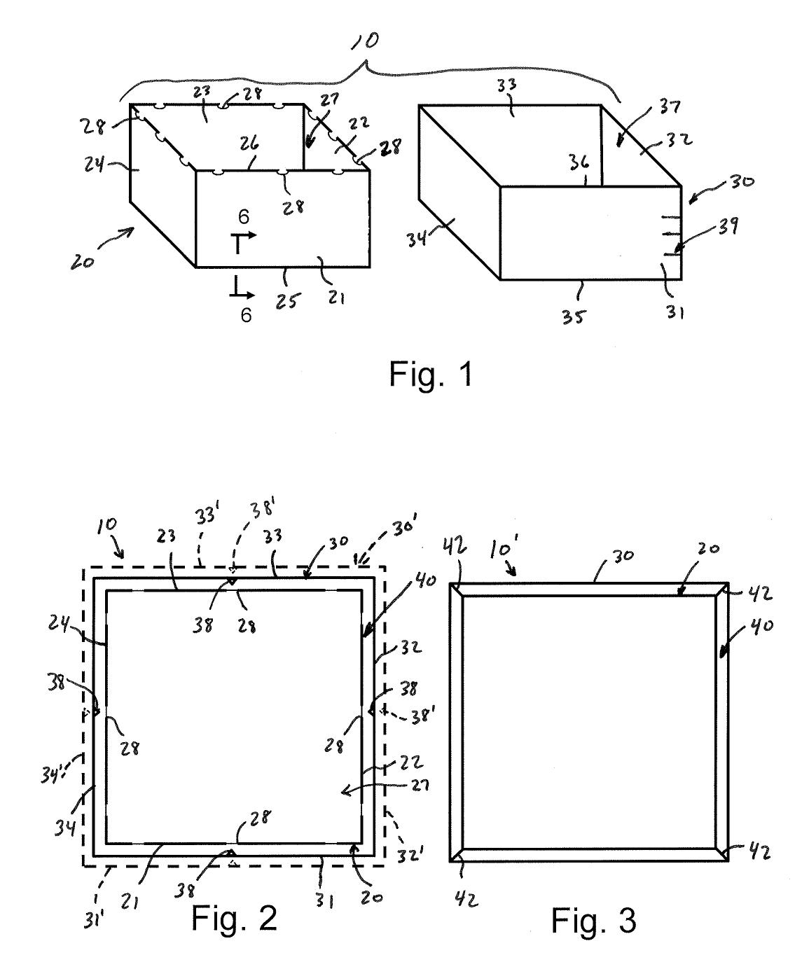

Referring to FIGS. 1, 2 and 6, a lumpectomy knife set 10 in accordance with an embodiment of the present disclosure will be described. While the knife set 10 is described herein with respect to a lumpectomy of breast tissue, it may be designed and utilized for other procedures on tissue having similar texture, density, and physical characteristics for which the push excision may be favored.

The knife set 10 includes a main knife 20 and a margin knife 30. In the present embodiment, the main knife 20 has four walls 21, 22, 23, 24 interconnected in a square configuration with a through opening 27. Each wall 21-24 is preferably planar and extends between a knife edge 25 and an upper edge 26. As illustrated in FIG. 6, in the present embodiment, the knife edge 25 has a symmetrically tapered, flat smooth configuration. The knife edge 25 may have other configurations, for example, serrated, asymmetrical, compound, chisel, convex or the like. In the illustrated embodiment, the upper edge 26 of each wall 21-24 includes a plurality of holes 28, hooks or the like, which are configured to receive and grasp sutures attached to the tissue. Such suture-grasp allows tissue elevation on the inner excised tissue, raising resection area to provide smoother cut. The number and position of holes 28 may be varied from that illustrated and may vary between the different walls 21-24.

The margin knife 30 has a configuration which complements that of the main knife 20. Accordingly, in the present embodiment, the margin knife 30 has four walls 31, 32, 33, 34 interconnected in a square configuration with a through opening 37 larger than an outer dimension of the main knife 20. Each wall 31-34 is preferably planar and extends between a knife edge 35 and an upper edge 36. As with the main knife 20, the knife edge 35 may have various configurations and may be the same or different than the configuration of the main knife edge 25, i.e. symmetrically tapered, flat smooth configuration. The knife edge 35 may have other configurations, for example, serrated, asymmetrical, compound, chisel, convex or the like.

Referring to FIG. 2, the margin knife 30 is positioned concentrically about the main knife 20 such that a margin gap 40 is defined therebetween. In the illustrated embodiment, each wall 31-34 of the margin knife 30 has an indexing mark 38 which aligns with a center hole 28 of a respective main knife wall 21-24 to facilitate concentric alignment of the margin knife 30 about the main knife 20. The main knife 20 may have different indexing marks than the holes 28. It is also contemplated that the main knife 20 and the margin knife 30 may be interconnected to one another such that the concentric alignment is fixed. For example, FIG. 3 illustrates an alternative embodiment of the knife set 10′ wherein connectors 42 extend between the main knife 20 and the margin knife 30, for example, in each corner, to fix the relationship between the knifes 20, 30. With this embodiment, the margin gap 40 is fixed.

Referring again to FIG. 2, the size of main knife 20 is selected such that the through opening 27 corresponds to the desired tumor excision size and the size of the margin knife 30 is selected such that the through opening 37 provides the desired margin gap 40 to achieve the desired thickness of resection marginal tissue. A knife set 10 may include multiple main knifes 20 and multiple margin knifes 30, 30′ to achieve the desired excision. Additionally, having multiple margin knifes 30, 30′, the thickness of the margin gap 40, and thereby the resection marginal tissue, may be increased by simply selecting a larger margin knife 30′. The walls 31′-34′ again have indexing marks 38′ to facilitate alignment of the margin knife 30′ with the main knife 20.

Once the main knife 20 has been positioned relative to the target tissue and the margin knife 30 positioned and aligned concentrically thereabout, the main knife 20 and the margin knife 30 are simultaneously pushed into the tissue with a single push action. The outer margin knife 30 may include an optional depth scale 39 which allows the surgeon to precisely push the knives 20, 30 to a desired depth. After the knives 20, 30 have been pushed to the desired depth, standard excision (lumpectomy) is carried out.

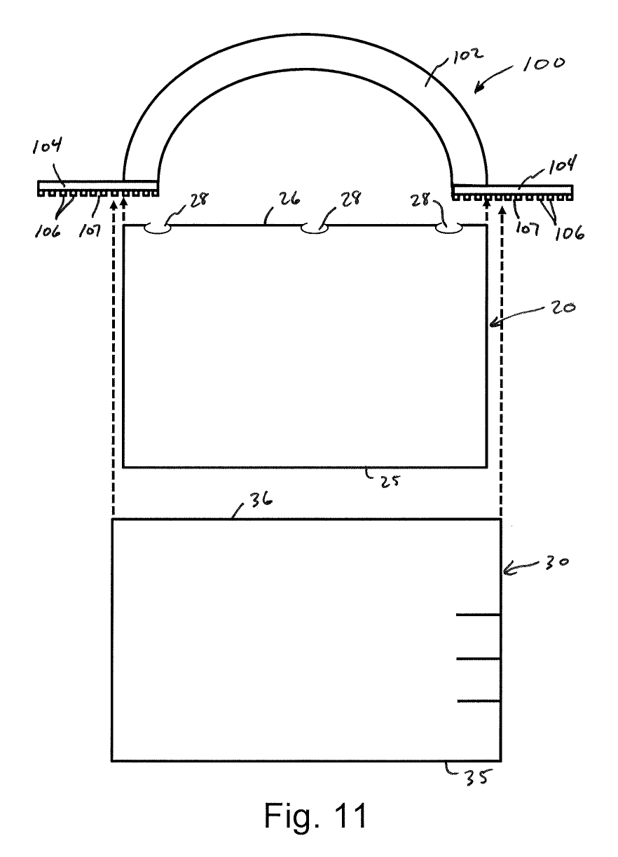

To facilitate simultaneous pushing, an optional handle assembly 100, as illustrated in FIG. 11, may be connected to the main knife 20 and the margin knife 30. In the illustrated embodiment, the handle assembly 100 includes a handle member 102 extending between a pair of connection platforms 104. Each of the connection platforms 104 includes a plurality of clips 106 extending therefrom with spaces 107 therebetween to receive the upper edges 26, 36 of the main knife 20 and margin knife 30. With the upper edges 26, 36 received in the spaces 107, the main knife 20 and the margin knife 30 are maintained in proper alignment and may be pushed simultaneously by pushing on the handle member 102. Other assemblies may alternatively be utilized to facilitate simultaneous pushing of the knives 20, 30.

The described push action of the knives 20, 30 is believed to provide a more precise, less-jagged, smoother excision of tumors and precise definition of the surrounding resection marginal tissue. It should be especially useful in obtaining precise, clean, non-jagged “shaves” (thin slices of tissue removed from the edge of the margin) and better margin pathological classification (R0, R1, or R2) for verification of removal of all malignant tissue in even very slim margins, which is the current trend. The clean shaves may also provide better stained tissue analysis versus rough or jagged stains. Additionally, the precise alignment between the linear knives 20, 30 allows for precise re-positioning, for example, if it is determined after the knives have been removed that a deeper excision is desired. Furthermore, the three-dimensional shape and minimal splicing movements minimize the challenge of properly orienting the excised specimen caused by the multiple splicing movements around all sides of the tumor using prior scalpels.

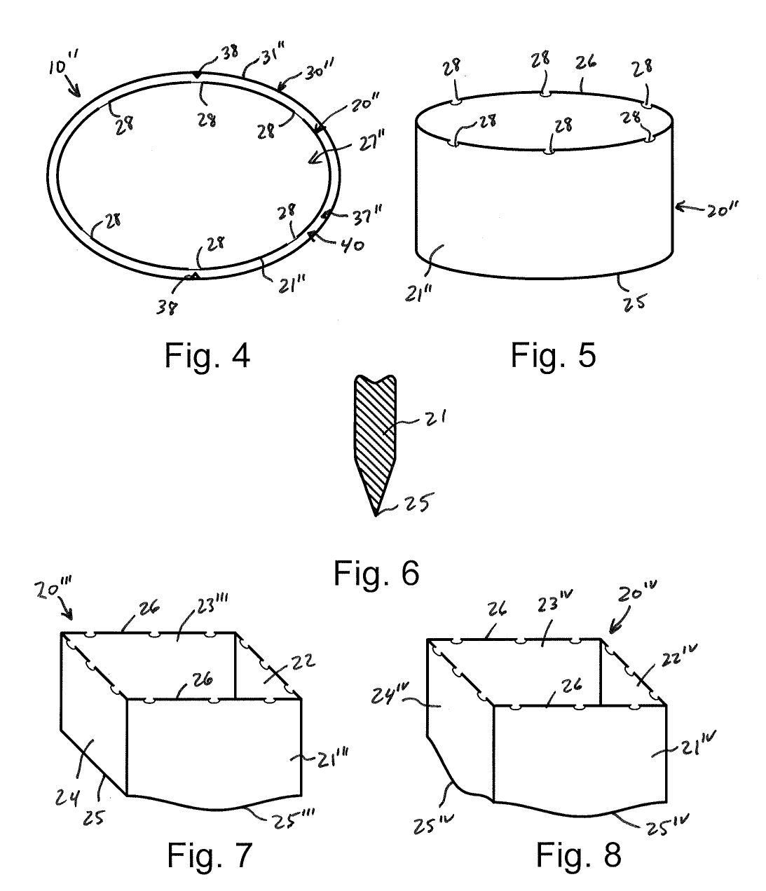

While the described knives 20, 30 have a square configuration, the present disclosure is not limited to such. Referring to FIGS. 4 and 5, a knife set 10″ having an oval configuration is illustrated. The main knife 20″ has a single wall 21″ having an oval configuration extending about an oval through opening 27″. As in the previous embodiment, the wall 21″ is preferably planar and extends between a knife edge 25 and an upper edge 26 and may include holes 28 along the upper edge 26. The margin knife 30″ has a complementary configuration with a single wall 31″ having an oval configuration extending about an oval through opening 37″. As in the previous embodiment, the wall 31″ is preferably planar and extends between a knife edge 35 and an upper edge 36 and has indexing marks 38 along the upper edge 36. The knives 20″, 30″ are again positioned concentrically to one another to define a margin gap 40. The knife set 10″ operates in the same manner as described above. The knives 20, 30 may have any other desired configuration, for example, rectangular, circular or other possibilities.

Referring to FIGS. 7 and 8, main knives 20′″, 20^{iv} in accordance with further embodiments of the present disclosure will be described. The main knife 20′″ in FIG. 7 is substantially the same as the main knife illustrated in FIG. 1 except that the front and rear walls 21′″, 23′″ have convex knife edges 25′″. The side walls 22, 24 have linear knife edges 25 as in the previous embodiment. The main knife 20^{iv} in FIG. 8 is substantially the same as the main knife illustrated in FIG. 1 except that each of walls 21^{iv}, 22^{iv}, 23^{iv}, 24^{iv} has convex knife edges 25^{iv}. The convex knife edges 25′″ allow the knife 20′″, 20^{iv} to be gently rocked back and forth to facilitate cutting, for example, if resistance is met while pushing down on the knife 20′″, 20^{iv}. The margin knife 30 paired with either of the knives 20′″, 20^{iv} may have similar knife edges 35 or may have different knife edges 35.

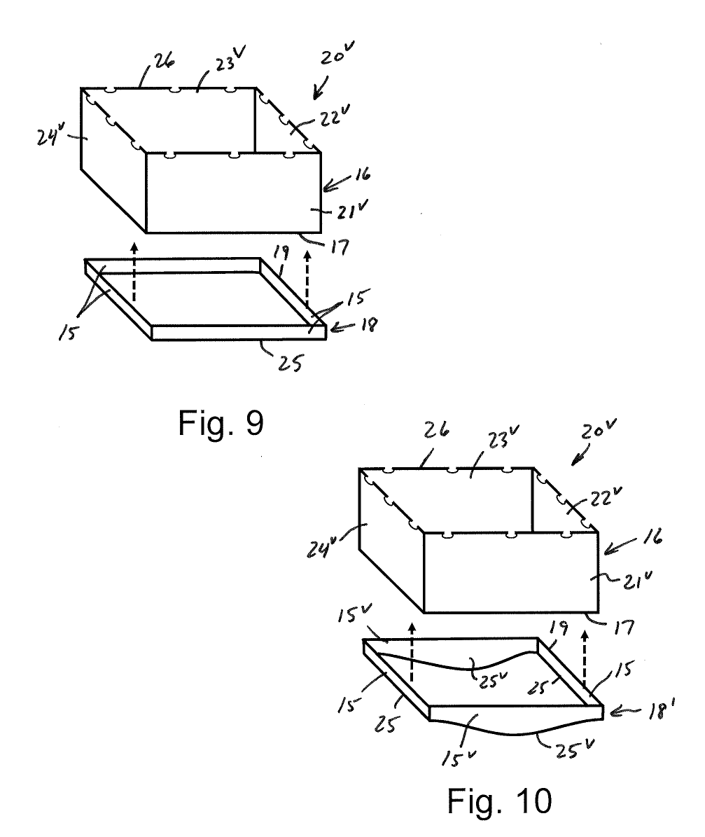

While each of the embodiments described herein have had an integral knife edge, it is contemplated that the knife edges may be connectable to a main body such that the knife edges are interchangeable and possibly disposable. Referring to FIGS. 9 and 10, an alternative main knife 20^{v} will described. The main knife 20^{v} includes a main body 16 defined by a plurality of walls 21^{v}-24^{v} which extend between an upper edge 26, similar to the previous embodiments, and a lower connector edge 17. The connector edge 17 is configured for connection to a blade assembly 18, 18′. The blade assembly 18 illustrated in FIG. 9 includes a plurality of walls 15, which complement the shape of the main body 16, with each wall 15 having a connector edge 19 and a knife edge 25. The blade assembly 18′ illustrated in FIG. 10 includes a plurality of walls 15, 15^{v}, which complement the shape of the main body 16, with each wall 15, 15^{v} having a connector edge 19, however, two of the walls 15^{v} have a convex knife edge 25^{v} and the other walls 15 have a linear knife edge 25. The knife edges 25, 25^{v} may have different blade configurations as described above. In each embodiment, the connector edge 19 is configured to removably connect with the connector edge 17 of the main body 16, for example, through a snap-fit connection. Once the blade assembly 18, 18′ is selected and attached to the main body 16, the main knife 20^{v} operates in the same manner as described above.

These and other advantages of the present disclosure will be apparent to those skilled in the art from the foregoing specification. Accordingly, it will be recognized by those skilled in the art that changes or modifications may be made to the above-described embodiments without departing from the broad inventive concepts of the present disclosure. It should therefore be understood that this disclosure is not limited to the particular embodiments described herein, but is intended to include all changes and modifications that are within the scope and spirit of the invention as defined in the claims.

What is claimed is:

1. A lumpectomy knife set comprising:
a main knife having at least one first planar wall extending between an upper edge and a knife edge, the at least one first planar wall defining a first geometric shape about a first through opening; and
a margin knife having at least one second planar wall extending between an upper edge and a knife edge, the at least one second planar wall defining a second geometric shape about a second through opening larger than the first through opening, the second geometric shape complementing the first geometric shape such that the margin knife is concentrically positionable about the main knife with a margin gap between the main knife and the margin knife.

2. The lumpectomy knife set according to claim 1, wherein the first and second geometric shapes are each selected from one of square, rectangular, oval and circular.

3. The lumpectomy knife set according to claim 1, wherein the main knife and the margin knife include index markings to facilitate concentric alignment therebetween.

4. The lumpectomy knife set according to claim 1, wherein the main knife and the margin knife are interconnected to one another by connectors extending within the margin gap.

5. The lumpectomy knife set according to claim 1 wherein the main knife and the margin knife are interconnected to one another by a handle assembly.

6. The lumpectomy knife set according to claim 5, wherein the handle assembly includes a handle member extending between a pair of connection platforms such that each of the connection platforms includes a plurality of clips extending therefrom with spaces therebetween to receive the upper edges of the main knife and the margin knife.

7. The lumpectomy knife set according to claim 1, wherein the at least one first planar wall of the main knife has a convex knife edge and the at least one second planar wall of the margin knife has a convex knife edge.

8. The lumpectomy knife set according to claim 1, wherein the knife edges are interchangeable.

9. The lumpectomy knife set according to claim 1, wherein the main knife includes a main body having a geometric shape defined by a plurality of walls configured to extend between an upper edge and a lower connector edge such that the lower connector edge is configured for connection to a blade assembly.

10. The lumpectomy knife set according to claim 9, wherein the blade assembly includes a plurality of walls which complement the shape of the main body with each wall having a connector edge and a knife edge such that the connector edge of each wall is configured to removably connect with the connector edge of the main body through a snap-fit connection.

11. The lumpectomy knife set according to claim 9, wherein the blade assembly includes a plurality of walls which complement the shape of the main body with each wall having a connector edge in which two of the walls have a convex knife edge and the other walls have a linear knife edge such that the connector edge of each wall is configured to removably connect with the connector edge of the main body through a snap-fit connection.
